(19) United States
(12) Patent Application Publication
(10) Pub. No.: US 2013/0078645 A1
(43) Pub. Date: Mar. 28, 2013

(54) BIOMARKERS

(71) Applicants: Sabine Bahn, Cambridge (GB); Marlis Huebner, Cambridge (GB)

(72) Inventors: Sabine Bahn, Cambridge (GB); Marlis Huebner, Cambridge (GB)

(73) Assignee: PSYNOVA NEUROTECH LTD., Cambridge (GB)

(21) Appl. No.: 13/575,692

(22) PCT Filed: Jan. 28, 2011
(86) PCT No.: PCT/GB2011/050136

§ 371 Date: Nov. 20, 2012

(30) Foreign Application Priority Data
Jan. 29, 2010 (GB) 1001444.7

(51) Int. Cl. G01N 33/68; C12Q 1/37; C12Q 1/48; C12Q 1/533

(52) U.S. Cl. CPC G01N 33/6896; C12Q 1/533; C12Q 1/37; C12Q 1/48
USPC 435/7.4; 435/4; 435/24; 435/15; 436/501; 435/23

(57) ABSTRACT

The invention relates to a method of diagnosing or monitoring schizophrenia or other psychotic disorder. The biomarkers used are selected from cyclophilin A, cytosalic non-specific dipepditase, Caoctosin-like protein, Glucose-6-phosphate isomerase, uncharacterized protein KIAA0423, myosin 14, myosin 15, nicotinamide phosphoribosyltransferase, pyruvate kinase isozyme R/L, phosphoglyterate mutase 4.

### FIELD OF THE INVENTION

The invention relates to a method of diagnosing or monitoring schizophrenia or other psychotic disorder.

### BACKGROUND OF THE INVENTION

Schizophrenia is a psychiatric diagnosis that describes a mental disorder characterized by abnormalities in the perception or expression of reality. It most commonly manifests as auditory hallucinations, paranoid or bizarre delusions, or disorganized speech and thinking with significant social or occupational dysfunction. Onset of symptoms typically occurs in young adulthood, with approximately 0.4-0.6% of the population affected. Diagnosis is based on the patient's self-reported experiences and observed behavior. No laboratory test for schizophrenia currently exists.

Studies suggest that genetics, early environment, neurobiology, psychological and social processes are important contributory factors; some recreational and prescription drugs appear to cause or worsen symptoms. Current psychiatric research is focused on the role of neurobiology, but no single organic cause has been found. Due to the many possible combinations of symptoms, there is debate about whether the diagnosis represents a single disorder or a number of discrete syndromes.

The disorder is thought to mainly affect cognition, but it also usually contributes to chronic problems with behavior and emotion. People with schizophrenia are likely to have additional (comorbid) conditions, including major depression and anxiety disorders; the lifetime occurrence of substance abuse is around 40%. Social problems, such as long-term unemployment, poverty and homelessness, are common. Furthermore, the average life expectancy of people with the disorder is 10 to 12 years less than those without, due to increased physical health problems and a higher suicide rate.

An important utility of biomarkers for psychotic disorders is their response to medication. Administration of antipsychotics remains a subjective process, relying solely on the experience of clinicians. Furthermore, the development of antipsychotic drugs has been based on chance findings often with little relation to the background driving the observations.

Schizophrenia is treated primarily with antipsychotic medications which are also referred to as neuroleptic drugs or neuroleptics. Newer antipsychotic agents such as Clozapine, Olanzapine, Quetiapine or Risperidone are thought to be more effective in improving negative symptoms of psychotic disorders than older medication like Chlorpromazine. Furthermore, they induce less extrapyramidal side effects (EPS) which are movement disorders resulting from antipsychotic treatment.

The history of neuroleptics dates back to the late 19th century. The flourishing dye industry catalyzed development of new chemicals that lay the background to modern day atypical antipsychotics. Developments in anti malaria, antihistamine and anaesthetic compounds also produced various neuroleptics. The common phenomenon to all these processes is a fundamental lack of understanding of the biological mechanisms and pathways that these drugs affect, apart from the observation that they prominently block D2 receptors in the striatum.

There is therefore a pressing need for objective molecular readouts that can diagnose schizophrenia or other psychotic disorders and furthermore indicate whether a patient is responding to medication, as well as for predicting prognosis.

### SUMMARY OF THE INVENTION

According to a first aspect of the invention, there is provided the use of one or more first analytes selected from: Cyclophilin A, Cytosolic non-specific dipeptidase, Coactosin-like protein, Glucose-6-phosphate isomerase, Uncharacterized protein KIAA0423, Myosin 14, Myosin 15, Nicotinamide phosphoribosyltransferase, Pyruvate kinase isozyme R/L and Phosphoglycerate mutase 4 as a biomarker for schizophrenia, or predisposition thereto.

According to a second aspect of the invention, there is provided the use of

Cyclophilin A, Pyruvate kinase isozyme R/L, Phosphoglycerate mutase 4, Glucose-6-phosphate isomerase and L-lactate dehydrogenase B as a specific panel of analyte biomarkers for schizophrenia or other psychotic disorder, or predisposition thereto.

According to a further aspect of the invention, there is provided a method of diagnosing or monitoring schizophrenia, or predisposition thereto, comprising detecting and/or quantifying, in a sample from a test subject, the analyte biomarkers defined herein.

According to a further aspect of the invention, there is provided a method of monitoring efficacy of a therapy in a subject having, suspected of having, or of being predisposed to schizophrenia, comprising detecting and/or quantifying, in a sample from said subject, the analyte biomarkers defined herein.

A further aspect of the invention provides ligands, such as naturally occurring or chemically synthesised compounds, capable of specific binding to the analyte biomarker. A ligand according to the invention may comprise a peptide, an antibody or a fragment thereof, or an aptamer or oligonucleotide, capable of specific binding to the analyte biomarker. The antibody can be a monoclonal antibody or a fragment thereof capable of specific binding to the analyte biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

A biosensor according to the invention may comprise the analyte biomarker or a structural/shape mimic thereof capable of specific binding to an antibody against the analyte biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the analyte. In these uses, the detection and/or quantification can be performed on a biological sample such as from the group consisting of CSF, whole blood, blood serum, plasma, urine, saliva, or other bodily fluid, breath, e.g. as condensed breath, or an extract or purification therefrom, or dilution thereof.

Diagnostic or monitoring kits are provided for performing methods of the invention. Such kits will suitably comprise a ligand according to the invention, for detection and/or quantification of the analyte biomarker, and/or a biosensor, and/or an array as described herein, optionally together with instructions for use of the kit.

A further aspect of the invention is a kit for monitoring or diagnosing schizophrenia, comprising a biosensor capable of detecting and/or quantifying the analyte biomarkers as defined herein.

Biomarkers for schizophrenia or other psychotic disorders are essential targets for discovery of novel targets and drug molecules that retard or halt progression of the disorder. As the level of the analyte biomarker is indicative of disorder and of drug response, the biomarker is useful for identification of novel therapeutic compounds in in vitro and/or in vivo assays. Biomarkers of the invention can be employed in methods for screening for compounds that modulate the activity of the analyte.

Thus, in a further aspect of the invention, there is provided the use of a ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the analyte in a subject, comprising administering a test substance to a subject animal and detecting and/or quantifying the level of the analyte biomarker present in a test sample from the subject.

### BRIEF DESCRIPTION OF THE FIGURES

FIG. 1: Principal Component Analysis (PCA) of differentially expressed cellular proteins in unstimulated and stimulated PBMCs. PCA of the differentially expressed proteins identified in unstimulated (A+C, n=5) and stimulated PBMCs (B, n=14) between AN patients and HC subjects. (A) The first two principal components account for 49.6% of the total variance. (B) The first two principal components account for 69.5% of the total variance. (C) PCA showing the degree of separation between AN and AT patients and HC subjects in unstimulated PBMCs.

FIG. 2: Overview of LC-MS^{E}-derived cellular proteins involved in glycolysis. Shown is the glycolysis pathway and the proteins detected by LC-MS^{E} in unstimulated (US) and stimulated (ST) PBMCs. Proteins were significantly increased (↑), decreased (↓) or unchanged (⇄) in AN patients compared to HC subjects. N/A=catalytic enzymes that were not detected.

FIG. 3: Example of prediction model built on a protein cluster comprised of GAPDH and GPI. Grey and black dots represent AN patient and HC subjects. Dots located in the pale and dark grey regions are predicted as schizophrenia and control, respectively. The boundary is determined by Fisher's discriminant analysis. Precision is calculated as the percentage of the dots that are red and located in the red regions. Prediction results for (A) randomly-selected training set and (B) the remaining half of the sample set (test set).

FIG. 4: Metabolic serum and cell markers in schizophrenia compared to healthy controls. Boxplots showing the mean±standard deviation of (A) circulating glucose and insulin levels in serum of 12 AN, 7 AT patients and 19 HC subjects, (B) the percentages of PBMCs expressing GLUT1 and the insulin receptor after 72 h stimulation with SEB+CD28 in 8 AN patients and 8 HC subjects and (C) lactate levels in PBMC supernatants after 72 h stimulation with SEB+CD28 of 8 AN patients and 8 HC subjects. *P<0.05, **P<0.01

### DETAILED DESCRIPTION OF THE INVENTION

The term “biomarker” means a distinctive biological or biologically derived indicator of a process, event, or condition. Analyte biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

It will be readily apparent to the skilled person that the analytes listed herein are known and have been described in the literature.

According to a first aspect of the invention, there is provided the use of one or more first analytes selected from: Cyclophilin A, Cytosolic non-specific dipeptidase, Coactosin-like protein, Glucose-6-phosphate isomerase, Uncharacterized protein KIAA0423, Myosin 14, Myosin 15, Nicotinamide phosphoribosyltransferase, Pyruvate kinase isozyme R/L and Phosphoglycerate mutase 4 as a biomarker for schizophrenia, or predisposition thereto.

In one embodiment of the first aspect of the invention, the first analyte is selected from Cyclophilin A, Pyruvate kinase isozyme R/L, Phosphoglycerate mutase 4 and Glucose-6-phosphate isomerase.

In a further embodiment, the first analyte is selected from Cyclophilin A. This particular biomarker is demonstrated to be the most statistically significant marker by data enclosed herein.

In one embodiment of the first aspect of the invention, the use additionally comprises one or more second analytes selected from: L-lactate dehydrogenase B, Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1 and Triosephosphate isomerase.

In one embodiment of the first aspect of the invention, the second analyte is selected from L-lactate dehydrogenase B.

According to a second aspect of the invention, there is provided the use of Cyclophilin A, Pyruvate kinase isozyme R/L, Phosphoglycerate mutase 4, Glucose-6-phosphate isomerase and L-lactate dehydrogenase B as a specific panel of analyte biomarkers for schizophrenia or other psychotic disorder, or predisposition thereto. This panel of biomarkers contains the most statistically significant marker identified in data described herein, namely Cyclophilin A. The panel also contains proteins associated with the glycolysis pathway, such as Pyruvate kinase isozyme R/L, Phosphoglycerate mutase 4, Glucose-6-phosphate isomerase and L-lactate dehydrogenase B, which the enclosed data demonstrates increased expression in first onset, antipsychotic naive schizophrenia patients.

According to one particular aspect of the invention which may be mentioned, there is provided the use of one or more first analytes selected from: Cytosolic non-specific dipeptidase, Coactosin-like protein, Glucose-6-phosphate isomerase, Uncharacterised protein KIAA0423, L-lactate dehydrogenase B, Myosin 14, Myosin 15, Nicotinamide phosphoribosyltransferase, Cyclophilin A, Pyruvate kinase isozyme R/L and Phosphoglycerate mutase 4 as a biomarker for schizophrenia or other psychotic disorder, or predisposition thereto.

In one embodiment of the one particular aspect of the invention, the first analyte is selected from Cytosolic non-specific dipeptidase, Glucose-6-phosphate isomerase and L-lactate dehydrogenase B.

In one embodiment of any of the aforementioned aspects of the invention, the first analyte is other than Glucose-6-phosphate isomerase.

In one embodiment of any of the aforementioned aspects of the invention, the first analyte is other than Phosphoglycerate mutase 4.

Thus, according to a further aspect of the invention, there is provided the use of one or more first analytes selected from: Cytosolic non-specific dipeptidase, Coactosin-like protein, Uncharacterised protein KIAA0423, L-lactate dehydrogenase B, Myosin 14, Myosin 15, Nicotinamide phosphoribosyltransferase, Cyclophilin A and Pyruvate kinase isozyme R/L as a biomarker for schizophrenia or other psychotic disorder, or predisposition thereto.

In one embodiment of any of the aforementioned aspects of the invention, the use additionally comprises one or more second analytes selected from: Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1 and Triosephosphate isomerase.

According to a further particular aspect of the invention which may be mentioned, there is provided the use of two or more second analytes selected from: Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1 and Triosephosphate isomerase as a biomarker for schizophrenia or other psychotic disorder, or predisposition thereto.

In one embodiment of any of the aforementioned aspects of the invention, the second analyte additionally comprises Glucose-6-phosphate isomerase.

In one embodiment of any of the aforementioned aspects of the invention, the second analyte additionally comprises Phosphoglycerate mutase 4.

Thus, according to a further aspect of the invention, there is provided the use of two or more second analytes selected from: Glucose-6-phosphate isomerase, Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1, Triosephosphate isomerase and Phosphoglycerate mutase 4 as a biomarker for schizophrenia or other psychotic disorder, or predisposition thereto.

In one embodiment of any of the aforementioned aspects of the invention, the second analyte is selected from Fructose bisphosphate aldolase, Glyceraldehyde-3-phosphate dehydrogenase, Phosphoglycerate kinase 1 and Triosephosphate isomerase.

Data is presented herein which identifies these 18 differentially expressed proteins between first onset, antipsychotic-naive patients and controls. Thus, according to a further aspect of the invention there is provided the use of Cytosolic non-specific dipeptidase, Coactosin-like protein, Glucose-6-phosphate isomerase, Uncharacterised protein KIAA0423, L-lactate dehydrogenase B,

Myosin 14, Myosin 15, Nicotinamide phosphoribosyltransferase, Cyclophilin A, Pyruvate kinase isozyme R/L, Phosphoglycerate mutase 4, Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1 and Triosephosphate isomerase as a specific panel of analyte biomarkers for schizophrenia or other psychotic disorder, or predisposition thereto.

Surprisingly, 7 of these proteins were associated with the glycolytic pathway and patient-control differences were more prominent in stimulated compared to unstimulated PBMCs. Thus, acccording to a further aspect of the invention there is provided the use of one or more proteins associated with the glycolytic pathway as a biomarker for schizophrenia or other psychotic disorder, or predisposition thereto. In one embodiment, the protein associated with the glycolytic pathway is selected from Cytosolic non-specific dipeptidase, Glucose-6-phosphate isomerase, L-lactate dehydrogenase B, Fructose bisphosphate aldolase, Glyceraldehyde-3-phosphate dehydrogenase, Phosphoglycerate kinase 1 and Triosephosphate isomerase. According to a further aspect of the invention, there is provided the use of Cytosolic non-specific dipeptidase, Glucose-6-phosphate isomerase, L-lactate dehydrogenase B, Fructose bisphosphate aldolase, Glyceraldehyde-3-phosphate dehydrogenase, Phosphoglycerate kinase 1 and Triosephosphate isomerase as a specific panel of analyte biomarkers for schizophrenia or other psychotic disorder, or predisposition thereto.

None of the analyte biomarkers identified herein were altered in chronically ill antipsychotic-treated patients. Non-linear multivariate statistical analysis showed that small subsets of these glycolytic proteins could be used as a signal for distinguishing first onset patients from controls with high precision. Functional analysis of PBMCs did not reveal any difference in glycolytic flux between patients and controls despite increased levels of the glucose transporter-1 (GLUT1) and decreased levels of the insulin receptor in patients. The inventors also found that the same subjects showed increased serum levels of insulin, consistent with the idea that some schizophrenia patients are insulin resistant. Therefore, the results presented herein show that schizophrenia patients respond differently to PBMC activation and this is manifested at disease onset and may be modulated by antipsychotic treatment. The altered glycolytic protein signature associated with this effect could therefore be of diagnostic and prognostic value.

In one embodiment, one or more of the biomarkers may be replaced by a molecule, or a measurable fragment of the molecule, found upstream or downstream of the biomarker in a biological pathway.

References herein to “other psychotic disorder” relate to any appropriate psychotic disorder according to DSM-IV Diagnostic and Statistical Manual of Mental Disorders, 4th edition, American Psychiatric Assoc, Washington, D.C., 2000. In one particular embodiment, the other psychotic disorder is a psychotic disorder related to schizophrenia. Examples of psychotic disorders related to schizophrenia include brief psychotic disorder delusional disorder, psychotic disorder due to a general medical condition, schizoeffective disorder, schizophreniform disorder, and substance-induced psychotic disorder.

As used herein, the term “biosensor” means anything capable of detecting the presence of the biomarker. Examples of biosensors are described herein.

Biosensors according to the invention may comprise a ligand or ligands, as described herein, capable of specific binding to the analyte biomarker. Such biosensors are useful in detecting and/or quantifying an analyte of the invention.

Diagnostic kits for the diagnosis and monitoring of schizophrenia or other psychotic disorder are described herein. In one embodiment, the kits additionally contain a biosensor capable of detecting and/or quantifying an analyte biomarker.

Monitoring methods of the invention can be used to monitor onset, progression, stabilisation, amelioration and/or remission.

In methods of diagnosing or monitoring according to the invention, detecting and/or quantifying the analyte biomarker in a biological sample from a test subject may be performed on two or more occasions. Comparisons may be made between the level of biomarker in samples taken on two or more occasions. Assessment of any change in the level of the analyte biomarker in samples taken on two or more occasions may be performed. Modulation of the analyte biomarker level is useful as an indicator of the state of schizophrenia or other psychotic disorder or predisposition thereto. An increase in the level of the biomarker, over time is indicative of onset or progression, i.e. worsening of this disorder, whereas a decrease in the level of the analyte biomarker indicates amelioration or remission of the disorder, or vice versa.

A method of diagnosis or monitoring according to the invention may comprise quantifying the analyte biomarker in a test biological sample from a test subject and comparing the level of the analyte present in said test sample with one or more controls.

The control used in a method of the invention can be one or more control(s) selected from the group consisting of: the level of biomarker analyte found in a normal control sample from a normal subject, a normal biomarker analyte level;

a normal biomarker analyte range, the level in a sample from a subject with schizophrenia or other psychotic disorder, or a diagnosed predisposition thereto; schizophrenia or other psychotic disorder biomarker analyte level, or schizophrenia or other psychotic disorder biomarker analyte range.

In one embodiment, there is provided a method of diagnosing schizophrenia or other psychotic disorder, or predisposition thereto, which comprises:
(a) quantifying the amount of the analyte biomarker in a test biological sample; and
(b) comparing the amount of said analyte in said test sample with the amount present in a normal control biological sample from a normal subject.

A higher level of the analyte biomarker in the test sample relative to the level in the normal control is indicative of the presence of schizophrenia or other psychotic disorder, or predisposition thereto; an equivalent or lower level of the analyte in the test sample relative to the normal control is indicative of absence of schizophrenia or other psychotic disorder and/or absence of a predisposition thereto.

The term “diagnosis” as used herein encompasses identification, confirmation, and/or characterisation of schizophrenia or other psychotic disorder, or predisposition thereto. By predisposition it is meant that a subject does not currently present with the disorder, but is liable to be affected by the disorder in time. Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing relapse rates.

Also provided is a method of monitoring efficacy of a therapy for schizophrenia or other psychotic disorder in a subject having such a disorder, suspected of having such a disorder, or of being predisposed thereto, comprising detecting and/or quantifying the analyte present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for schizophrenia or other psychotic disorder in a subject, comprising:
(a) quantifying the amount of the analyte biomarker; and
(b) comparing the amount of said analyte in said test sample with the amount present in one or more control(s) and/or one or more previous test sample(s) taken at an earlier time from the same test subject.

A decrease in the level of the analyte biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-psychotic therapy. Samples can be taken at intervals over the remaining life, or a part thereof, of a subject.

The term “detecting” as used herein means confirming the presence of the analyte biomarker present in the sample. Quantifying the amount of the biomarker present in a sample may include determining the concentration of the analyte biomarker present in the sample. Detecting and/or quantifying may be performed directly on the sample, or indirectly on an extract therefrom, or on a dilution thereof.

In alternative aspects of the invention, the presence of the analyte biomarker is assessed by detecting and/or quantifying antibody or fragments thereof capable of specific binding to the biomarker that are generated by the subject's body in response to the analyte and thus are present in a biological sample from a subject having schizophrenia or other psychotic disorder or a predisposition thereto.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the analyte biomarker in the sample or samples. Biological samples that may be tested in a method of the invention include cerebrospinal fluid (CSF), whole blood, blood serum, plasma, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Detection and/or quantification of analyte biomarkers may be performed by detection of the analyte biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample of cerebrospinal fluid (CSF) by SELDI TOF or MALDI TOF to detect the presence or level of the analyte biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the analyte biomarkers may be performed using an immunological method, involving an antibody, or a fragment thereof capable of specific binding to the analyte biomarker. Suitable immunological methods include sandwich immunoassays, such as sandwich ELISA, in which the detection of the analyte biomarkers is performed using two antibodies which recognize different epitopes on a analyte biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of analyte biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for an analyte biomarker of the invention may be immobilised on an avidin or streptavidin surface, the immobilised ligand may then be exposed to a sample containing or suspected of containing the analyte biomarker in order to detect and/or quantify an analyte biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab')₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e. g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing or monitoring schizophrenia or other psychotic disorders which comprises a biosensor, microanalytical, microengineered, microseparation and/or immunochromatography system configured to detect and/or quantify any of the biomarkers defined herein.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal.

Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Methods involving detection and/or quantification of one or more analyte biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-psychotic disorder therapeutic; or the test substance can be novel synthetic or natural chemical entity, or a combination of two or more of the aforesaid substances.

There is provided a method of identifying a substance capable of promoting or suppressing the generation of the analyte biomarker in a subject, comprising exposing a test cell to a test substance and monitoring the level of the analyte biomarker within said test cell, or secreted by said test cell.

The test cell could be prokaryotic, however a eukaryotic cell will suitably be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing the analyte.

Screening methods also encompass a method of identifying a ligand capable of binding to the analyte biomarker according to the invention, comprising incubating a test substance in the presence of the analyte biomarker in conditions appropriate for binding, and detecting and/or quantifying binding of the analyte to said test substance.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of inhibiting, directly or indirectly, the activity of the analyte biomarker, or of suppressing generation of the analyte biomarker. The term “substances” includes substances that do not directly bind the analyte biomarker and directly modulate a function, but instead indirectly modulate a function of the analyte biomarker. Ligands are also included in the term substances; ligands of the invention (e.g. a natural or synthetic chemical compound, peptide, aptamer, oligonucleotide, antibody or antibody fragment) are capable of binding, suitably specific binding, to the analyte.

The invention further provides a substance according to the invention for use in the treatment of schizophrenia or other psychotic disorder, or predisposition thereto.

Also provided is the use of a substance according to the invention in the treatment of schizophrenia or other psychotic disorder, or predisposition thereto.

Also provided is the use of a substance according to the invention as a medicament.

Yet further provided is the use of a substance according to the invention in the manufacture of a medicament for the treatment of schizophrenia or other psychotic disorder, or predisposition thereto.

A kit for diagnosing or monitoring schizophrenia or other psychotic disorder, or predisposition thereto is provided. Suitably a kit according to the invention may contain one or more components selected from the group: a ligand specific for the analyte biomarker or a structural/shape mimic of the analyte biomarker, one or more controls, one or more reagents and one or more consumables; optionally together with instructions for use of the kit in accordance with any of the methods defined herein.

The identification of biomarkers for schizophrenia or other psychotic disorder permits integration of diagnostic procedures and therapeutic regimes. Currently there are significant delays in determining effective treatment and hitherto it has not been possible to perform rapid assessment of drug response. Traditionally, many anti-psychotic therapies have required treatment trials lasting weeks to months for a given therapeutic approach. Detection of an analyte biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients at high risk of developing schizophrenia or other psychotic disorder. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder, poor patient compliance or substance abuse. If pharmacological treatment is assessed to be inadequate, then therapy can be reinstated or increased; a change in therapy can be given if appropriate. As the biomarkers are sensitive to the state of the disorder, they provide an indication of the impact of drug therapy or of substance abuse.

The following study illustrates the invention.

The aim of this study was to establish proteomic signatures using liquid chromatography mass spectrometry (LC-MS^{E}) profiling for unstimulated and stimulated peripheral blood mononuclear cells (PBMCs) isolated from first onset antipsychotic-naïve (AN) schizophrenia patients. Unstimulated PBMCs from antipsychotic-treated (AT) chronically ill schizophrenia patients were also investigated by LC-MS^{E} in order to determine which markers may be normalised by treatment and which may be indicators of underlying disease state. To investigate peripheral metabolic and immunological alterations associated with the onset of disease, PBMCs from AN patients were also stimulated in vitro, which results in activation of various signalling cascades associated with the immune response including the triggering of metabolic pathways (Fox C J et al, 2005; 5(11):844-52), and then analyzed using LC-MS^{E} profiling. The resulting proteomic fingerprints were characterised by functional analysis in silico and validated by mechanism of action studies in vitro.

### Methodology

Study Population and Demographics

The study was approved by the local ethics committee and conducted from 2007 to 2009 at the University Hospital of Cologne. Subjects comprised 12 first onset antipsychotic-naive (AN) patients suffering from first-episode paranoid psychosis (DSM-IV: 295.30) and 7 chronically ill antipsychotic-treated (AT) patients (DSM-IV: 295.30), as well as 19 healthy controls (HC) with no family history of schizophrenia or detectable medical, psychiatric or neurological history (Table 1).

TABLE 1
Demographic details of study cohorts

| | PBMCs | | PBMCs | |
|---|---|---|---|---|
| Demographic | (LC-MS^{E}) | | (validation) | |
| parameter | HC | SZ | HC | SZ |
| Number (n) | n = 19 | n = 19 | n = 13 | n = 15 |
| Age (y)* | 34.5 ± 7.2 | 29.7 ± 8.9 | 31.6 ± 12.0 | 30.0 ± 10.4 |
| Type (AN/AT) | N/A | 12/7 | N/A | 8/7 |
| Gender (m/f) | 12/7 | 14/5 | 5/8 | 6/9 |
| BMI* | 23.4 ± 3.0 | 23.8 ± 2.7 | 22.6 ± 2.5 | 24.2 ± 2.1 |
| Smoking (yes/no) | 7/12 | 9/10 | 10/3 | 10/5 |
| Cannabis (yes/no) | 16/3 | 13/6 | 10/3 | 8/7 |

*(mean ± SD)
Abbreviations:
PBMCs, peripheral blood mononuclear cells;
HC, healthy control;
SZ, schizophrenia patients;
AN, first-onset, antipsychotic-naive patients;
AT, antipsychotic-treated, chronically ill patients;
SD, standard deviation;
m, male;
f, female;
y, years;
BMI, body mass index

HCs were matched for age, gender, smoking, ethnicity, cannabis use, body mass index (BMI) and education. Psychopathology was assessed on the day of blood withdrawal. In addition, a validation cohort comprising 8 AN, 7 AT schizophrenia patients and 13 HC subjects was recruited. All participants were screened for medical disorders such as diabetes, heart disease, thyroid disease, autoimmune disease, recent infections or current or previous psychiatric illnesses using DSM-IV criteria and gave written informed consent.

PBMC and Serum Preparation

Blood was collected into 9 mL EDTA S Monovette tubes (Sarstedt; Leicester, UK). PBMCs were isolated by density gradient centrifugation at 750 g for 20 minutes using Ficoll-Paque Plus (GE Healthcare; Amersham, UK) and washed in Dulbecco's phosphate buffered saline (DPBS) (Invitrogen; Paisley, UK). Cells were stored in 90% foetal calf serum (FCS; Sigma; Dorset, UK) and 10% dimethyl sulfoxide (DMSO; Sigma) in liquid nitrogen prior to use. For serum, blood was collected into 7.5 mL S-Monovette tubes (Sarstedt). The tubes were placed at room temperature for 2 hours for blood coagulation, centrifuged at 4,000 g for 5 minutes and the supernatants stored at −80 ° C. prior to use.

PBMC Stimulation

PBMCs (1×10⁷) were thawed in RPMI-1640 medium (Sigma) supplemented with 10% FCS, 1% glutamine, penicillin, streptavidine and 1% DNAse (Sigma). For unstimulated PBMCs, cells were washed immediately in DPBS and stored as pellets at −80° C. prior to fractionation. For stimulated PBMCs (AN=8, HC=8), cells were kept in the thawing medium over night at 37° C. under 5% CO₂. The following morning, 7×10⁶ cells were stimulated with 1 μg/mL staphylococcal enterotoxin B (SEB; Sigma) and 1 μg/mL CD28 (BD Bioscience; Oxford, UK) in the thawing medium without DNAse for 72 h at 37° C. under 5% CO₂. Cell supernatants and pellets were collected after centrifugation at 15 000 g for 4 min. The pellets were washed twice with ice-cold DPBS and stored at −80° C. prior to use.

Subcellular Fractionation

Subcellular fractions were prepared from unstimulated and stimulated PBMC pellets along with quality control samples (n=8) aliquoted from a single donor. Protein intensity measurements were used to assess variability of the preparation, fractionation and mass spectrometric stages of the procedure. PBMC cytosolic fractions were produced using the ProteoExtract® Subcellular Proteome Extraction Kit according to the manufacturer's specifications (Merck; Darmstadt, Germany). The resulting subcellular composition was 71% and 59% soluble proteins in the unstimulated and stimulated samples as determined by Swiss-Prot annotation. Protein concentrations were measured using the BioRad DC™ Protein Assay (BioRad; Hercules, Calif., USA). Proteins were digested using the ProteoExtract® All-In-One Trypsin Digestion Kit (Merck) according to the manufacturer's protocol with minor changes. In brief, 4 μL trypsin (Promega, Southampton, UK) was added to samples after addition of blocking agent and samples were incubated for 17 h at 37° C. with shaking. The reactions were terminated by addition of 1.1 μL 8.8 M HCl (Sigma) and samples stored at −80° C. until mass spectrometry analysis.

Liquid Chromatography-Mass Spectrometry (LC-MS^{E}) and Data Analysis

The LC-MS^{E} profiling study was carried out and data acquired as described previously (Levin et al, Journal of Separation Science 2007; 30(14):2198-203). Resulting data were processed with the Waters ProteinLynx Global Server software v2.3 and searched against the human SwissProt v55 protein database (SIB Switzerland) as described previously (Levin et al, supra). The total ion current was used for data normalization. The mean intensity coefficient of variation of all proteins detected in the cytosolic fraction of quality control samples was 29%. Processed LC-MS^{E} data were exported to the software package R (http://cran.r-project.org) for filtering and protein intensities were calculated based on methods described previously (Levin et al, supra). In brief, criteria for inclusion required the appearance of a peptide in at least two out of three injections per sample and in at least 80% of samples in any of the groups. Calculation of protein abundance was based on correlating peptides with a cut-off set to 0.4 (Pearson's correlation)(Schwarz et al, J September Sci 2007 September; 30(14):2190-7). Standard statistical methods were used to investigate data structure and to test for potential experimental artefacts, the need for transformation or exclusion of outlying data. Student's t-test was applied to identify differentially expressed proteins (p<0.05). SIMCA-P+ 10.5 (Umetrics; Umea, Sweden) was used for principal component analysis (PCA) to determine the degree of overlap across the groups.

In Silico Pathway Analysis

For functional categorization and pathway analysis, statistically significant proteins were analyzed in silico using the Ingenuity Pathway Knowledgebase (IPKB) software. Assignment of functions and canonical pathways were performed automatically by computational algorithms as described previously (Liu et al, PROTEOMICS 2008; 8(3):582-603).

Protein Cluster Analysis

Protein cluster analysis is a useful computational technique that identifies determinants of a disease which have impact through cooperative function. Here, the LC-MS^{E} protein expression results were subjected to factor analysis (FA) to reduce the multi-dimensional data to the factors which have the highest influence on data structure by considering variance and noise. All combinations of these proteins were tested in simulations to identify those that give the greatest separation between patients and controls. The precision of each combination of analytes in separating patients and controls was tested through a corresponding kernel PCA prediction model. Similar to standard PCA, kernel PCA provides a new projection basis that yields maximal variance in descending order by performing eigenvalue decomposition on the data covariance matrix (Schoelkopf et al, Neural Computation 1998; 10(5): 1299-319). The prediction boundary of a kernel PCA projection is determined using Fisher's discriminant analysis (FDA). To assess prediction power, each kernel PCA model was built on a randomly selected training set consisting of half patient and half control sample data and then tested on a set consisting of the remaining data.

Validation Studies

Immunoblot Analysis

Differential expression of selected proteins identified by LC-MS^{E} analysis were tested further by immunoblot analysis using soluble PBMC fractions prepared from a separate validation cohort (5 AN schizophrenia, 5 AT schizophrenia and 5 HC subjects; Table 1) and primary antibodies against the proteins listed in Table 3. All antibodies were purchased from Abcam, Cambridge, UK. Detection and quantification were performed using the Odyssey Infrared Imaging System (LI-COR; Cambridge, UK). Intensities of immunoreactive protein bands were normalized to those of the β3-tubulin immunoreactivity in each track.

Insulin/Glucose

Assays were performed by the NIHR Cambridge Biomedical Research Centre, Core Biochemistry Assay Laboratory, Addenbrooke's Hospital and the necessary reagents and calibrants provided as described previously (Libby P. The American Journal of Medicine 2008; 121(10, Supplement 1):S21-S31). In brief, glucose levels were determined spectrophotometrically in 25 μL serum obtained from 12 AN, 8 AT schizophrenia and 19 HCs (same subjects used for PBMC LC-MS^{E} profiling study) using an adaptation of the hexokinase-glucose-6-phosphate dehydrogenase method on a Dimension RXL Clinical Chemistry System (Dade Behring; Milton Keynes, UK). Insulin was determined in 25 μL of serum obtained from the same subjects using a two-step time resolved fluorometric assay from Perkin Elmer (Beaconsfield, Bucks, UK).

Lactate Measurement

Lactate concentrations were measured in supernatants of stimulated PBMCs using an assay kit (Biovision; Mountain View, USA). In brief, 50 μL of cell supernatants were transferred in duplicate to a 96-well flat-bottom plate. Reaction mix buffer (50 μL; lactate enzyme and substrate) was added to the supernatants and incubated for 30 min at room temperature. The results were quantified at 450 nm using a plate reader (BioRad; Birmingham, UK).

Hexokinase Activity

Hexokinase activity (Sigma) was measured in supernatants of lysed PBMCs obtained from the validation cohort (8 AN schizophrenia and 8 HC subjects; Table 1). Cell pellets were resuspended in 230 μL homogenization buffer (150 mM KCl, 5 mM MgCl₂, 5 mM EDTA, 5 mM β-mercaptoethanol), incubated on ice for 30 min and centrifuged at 13,000 g for 5 min. In a spectrophotometer cuvette, 2.28 mL of Tris/MgCl₂ buffer, 0.5 mL 0.67 M glucose, 0.1 mL 16.5 mM ATP, 0.1 mL 6.8 mM NAD and 0.01 mL G6PD were mixed and preheated at 30° C. for 6 min, followed by addition of 0.1 mL of the cell supernatants. Hexokinase activity was based on reduction of NAD+ in the presence of G6PD and determined spectrophotometrically by recording the increase in absorbance at 340 nm over 10 min.

Cell Surface Markers

Stimulated PBMCs (1×10⁶) from the validation set (8 AN schizophrenia and 8 HC subjects; Table 1) were labelled with glucose transporter 1 (GLUT1) antibody conjugated to fluorescein isothiocyanate (FITC; R&D Systems; Abington, UK) and insulin receptor (IR) antibody conjugated to phycoerythrin (PE; BD Bioscience) in DPBS supplemented with 2% FCS (staining buffer). Reactions were incubated for 20 min at 4° C. in the dark and cells washed twice in staining buffer by centrifugation at 1,500 rpm for 3 min. Samples were analysed on the CyAn ADP Flow Cytometer.2 equipped with Summit v.4 software (Dako Cytomation; Copenhagen, Denmark). The percentages of cells expressing GLUT1 or IR were determined using FlowJo software (Tree Star; Ashland, Oreg., USA).

Statistical Analysis

Statistical analysis for all functional validation assays was performed using Student's t-test in Prism v.5 (GraphPad Software; La Jolla, Calif., USA). P-values of P<0.05 were considered significant.

### Results

PBMC Proteome Profiling

PBMCs isolated from AN (n=12) and AT (n=7) schizophrenia subjects and healthy controls (HC=19), consisted of >75% lymphocytes as determined by flow cytometric analysis. No differences in subpopulations of T cells, NK cells, B cells and monocytes were found between schizophrenia patients and HC subjects (data not shown). PBMCs were subjected to subcellular fractionation to enrich soluble proteins. In the unstimulated condition PMBCs from all volunteers were analysed on the LC-MS^{E} whereas in the stimulated condition only PBMCs from AN patients and HCs were analysed as the primary aim was to identify differentially expressed proteins in the first stages of the disease to eliminate medication effects. LC-MS^{E} analysis identified 5141 and 7713 peptides in unstimulated and stimulated PBMCs which translated to 185 and 441 non-redundant proteins, respectively, using the Swiss-Prot database. In total, 18 differentially expressed proteins were identified. Of these, only 6 proteins were altered in unstimulated PBMCs, 13 proteins were altered after stimulation with SEB+CD28, and one protein (lactate dehydrogenase B; LDHB) was altered in both conditions (Table 2).

TABLE 2
Differentially expressed cellular proteins between antipsychotic-naïve
patients and healthy controls
PBMC soluble extracts from AN patients and HC subjects were
analyzed by LC-MS^{E}. Accession number (Acc), protein
identification, P-value and fold change (AN/HC) are indicated for
differentially expressed proteins identified in either unstimulated
or stimulated PBMCs

| | | | | |
|---|---|---|---|---|
| | | | | Fold |
| | | | | change |
| | | | P- | (AN/ |
| Acc | Gene | Description | value^{#} | HC) |

Unstimulated PBMCs

| | | | | |
|---|---|---|---|---|
| Q96KP4 | CNDP2 | Cytosolic non-specific | .005 | 1.2 |
| | | dipeptidase | | |
| Q14019 | COTL1 | Coactosin-like protein | .02 | −1.2 |
| P06744 | GPI | Glucose-6-phosphate | .05 | −1.2 |
| | | isomerase | | |
| P54652 | HSP72 | Heat shock 70 kDa protein | .02 | −1.4 |
| Q9Y4F4 | K0423 | Uncharacterized protein | .003 | 1.1 |
| | | KIAA0423 | | |
| P07195* | LDHB | L-lactate dehydrogenase B | .04 | 1.2 |

Stimulated PBMCs

| | | | | |
|---|---|---|---|---|
| P09972 | ALDOC | Fructose bisphosphate | .01 | 1.2 |
| | | aldolase | | |
| P10809 | CH60 | 60 kDa heat shock protein | .04 | −1.4 |
| P04406 | GAPDH | Glyceraldehyde-3-phosphate | .03 | 1.2 |
| | | dehydrogenase | | |
| P61978 | HNRPK | Heterogeneous nuclear | .01 | 1.3 |
| | | ribonucleoprotein | | |
| P07195* | LDHB | L lactate dehydrogenase B | .02 | 1.3 |
| Q7Z406 | MYH14 | Myosin 14 | .04 | 1.2 |
| Q9Y2K3 | MYH15 | Myosin 15 | .02 | 1.2 |
| P43490 | NAMPT | Nicotinamide | .04 | 1.3 |
| | | phosphoribosyltransferase | | |
| P00558 | PGK1 | Phosphoglycerate kinase 1 | .003 | 1.4 |
| P62937 | PPIA | Cyclophilin A | <.001 | 1.4 |
| P60174 | TPIS | Triosephosphate isomerase | .008 | 1.5 |
| P30613 | PKLR | Pyruvate kinase isozyme R/L | .05 | 1.2 |
| Q8N0Y7 | PGAM4 | Phosphoglycerate mutase 4 | .05 | 1.2 |

^{#}Student's t-test
*Proteins were differentially expressed in unstimulated and stimulated fractions Note that all proteins, with the exception of CH60, showed increased expression in stimulated PBMCs from AN patients compared to those from HC subjects. None of the proteins that showed significant differences in expression between unstimulated PBMCs from AN and HC subjects were altered in AT patients with the exception of coactosin-like protein (COTL1) (p = 0.03; data not shown).

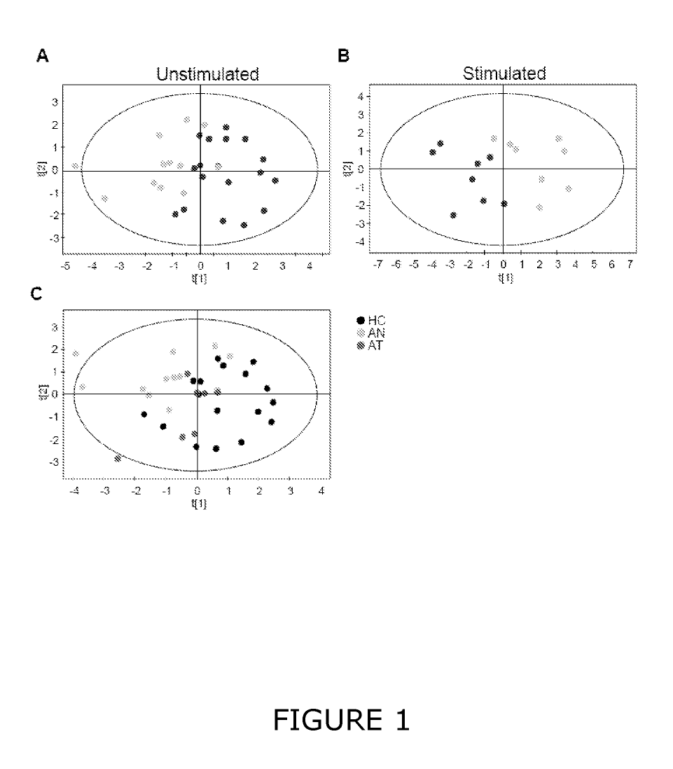

Principal component analysis (PCA) was employed to determine whether a separation according to diagnostic group can be achieved based on the differentially expressed proteins. PCA reduces multidimensional datasets by performing spectral decomposition analysis on covariance matrices. The first two dimensions (principal components 1 and 2) were considered that represent the greatest variances of the dataset (Davies AMC, Fearn T. Spectrosc Europe 2005; 4(17):20-3). AN patients showed good separation from HC subjects for unstimulated PBMCs and a greater separation for stimulated PBMCs (FIG. 1). In contrast, AT patients differed from AN patients in that they clustered more closely with the HC subjects.

Immunoblot Validation of Differentially Expressed Proteins

Selected biomarker candidates identified by LC-MS^{E} analysis were measured by immunoblot analysis using an independent PBMC sample cohort comprised of 5 AN and 5 AT patients as well as 5 HC subjects (Table 1). Reproducibility was assessed by comparing the fold changes obtained from the LC-MS^{E} and immunoblot analyses (Table 3).

In unstimulated PBMC samples, increased expression levels of cytosolic non-specific dipeptidase 2 (CNDP2) and LDHB were confirmed when comparing AN patients with HC subjects. Expression levels of these proteins did not differ between AT patients and HC subjects, consistent with the LC-MS^{E} results. In contrast, decreased expression levels of glucose 6-phosphate isomerase (GPI) in AN patients was not confirmed by immunoblot analysis (Table 3). In stimulated PBMC samples, increased expression of triosephosphate isomerase (TPIS), glyceraldehyde-3-phosphate dehydrogenase (GAPDH) and LDHB was confirmed by immunoblot analysis although that of aldolase C (ALDOC) and phosphoglycerate kinase 1 (PGK1) was not.

Characterisation of Differentially Expressed Proteins in Silico

Ingenuity Pathway Analysis

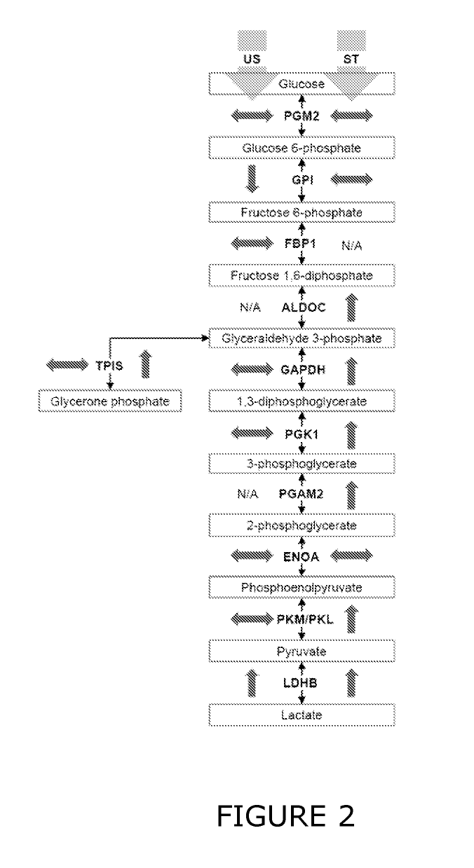

The Swiss-Prot accession codes of differentially expressed proteins were uploaded into the IPKB database to obtain information on relevant biological functions. The most significant (p=9.46E-04) canonical pathway associated with the differentially expressed proteins (GPI, LDHB) in unstimulated PBMCs was glycolysis. The same pathway was also the most significant (p=1.6E-11) for stimulated PBMCs which was associated with a higher number of differentially expressed proteins [(LDHB, PGK1, ALDOC, TPIS, GAPDH, PGAM4, Pyruvate kinase isozyme R/L (PKLR) FIG. 2)]. ALDOC and TPIS were also significantly associated with the fructose/mannose (p=5.32E-04) and inositol (p=1.05E-03) metabolism.

Protein Cluster analysis

To investigate the potential differential effects on glycolysis further, a nonlinear multivariate statistical approach was applied to identify combinations of glycolytic proteins that have high precision for distinguishing patients from controls. The top 20 protein combinations producing the best prediction models for separating AN patients from HC subjects were identified. The median precision prediction values were obtained after 1000 simulation tests for all combinations to avoid biased predictions associated with small sample sizes. The clusters generally gave lower prediction values for unstimulated compared to stimulated PBMCs, supporting the case that glycolysis is linked to immune response (Table 4).

TABLE 4
Median precision prediction values of the top 20 protein combinations

| Stimulated PBMCs | | Unstimulated PBMCs | |
|---|---|---|---|
| | Median | | Median |
| | precision | | precision |
| Protein clusters | (%) | Protein clusters | (%) |
| GAPDH + GPI | 80 (40-50)* | ENO1 + TPIS | 60-70 |
| ENO1 + GAPDH | 80 (50)* | ENO1 + PGK1 + TPIS | 50-60 |
| ENO1 + GAPDH + TPIS | 80 (50)* | ENO1 + GPI | 50-60 |
| ENO1 + GAPDH + PKM2 + TPIS | 80 (40)* | ENO1 + PKM2 + TPIS | 50 |
| ENO1 + GAPDH + GPI + PGM2 + TPIS | 80 (40-50)* | ENO1 + PKM2 | 50 |
| GAPDH + LDHA | 70-80 | ENO1 + LDHA + TPIS | 50 |
| GAPDH + GPI + PKM2 | 70-80 | ENO1 + LDHA + PGK1 + PKM2 | 50 |
| GAPDH + TPIS | 70-80 | ENO1 + FBP1 + GPI | 50 |
| GAPDH + LDHA + PKM2 + TPIS | 70-80 | ENO1 + GAPDH | 50 |
| ENO1 + GAPDH + PGM2 + TPIS | 70-80 | ENO1 + FBP1 + GPI + PGK1 | 50 |
| ENO1 + GAPDH + PGM2 + PKM2 + TPIS | 70-80 | ENO1 + GPI + PGK1 | 50 |
| ENO1 + GAPDH + LDHA | 70-80 | ENO1 + PGK1 | 50 |
| GAPDH + GPI + LDHA | 70-80 | ENO1 + LDHA + PGM2 | 50 |
| GAPDH + PGM2 + TPIS | 60-70 | GPI + TPIS | 40-50 |
| GAPDH + LDHA + PKM2 | 60-70 | GAPDH + GPI + TPIS | 40-50 |
| ENO1 + GAPDH + LDHA + PKM2 + TPIS | 70-80 | GPI + LDHA + PGM2 | 40-50 |
| GAPDH + PGM2 + TPIS | 60-70 | ENO1 + FBP1 + GPI + PKM2 | 40-50 |
| GAPDH + LDHA + PKM2 | 60-70 | FBP1 + GPI | 40-50 |
| GPI + PKM2 | 60-70 | ENO1 + PKM2 + PGM2 | 40-50 |
| GAPDH + PGM2 + PKM2 | 60 | ENO1 + PKM2 + PGM2 + TPIS | 40-50 |

Abbreviations:
ENO1, Enolase 1;
PKM2, Pyruvate kinase type M2;
PGM2, Phosphoglucomutase 2;
FBP, Fructose 1,6-bisphosphate;
N/A, not applicable
*Values were obtained by applying the corresponding protein cluster to the unstimulated PBMCs.

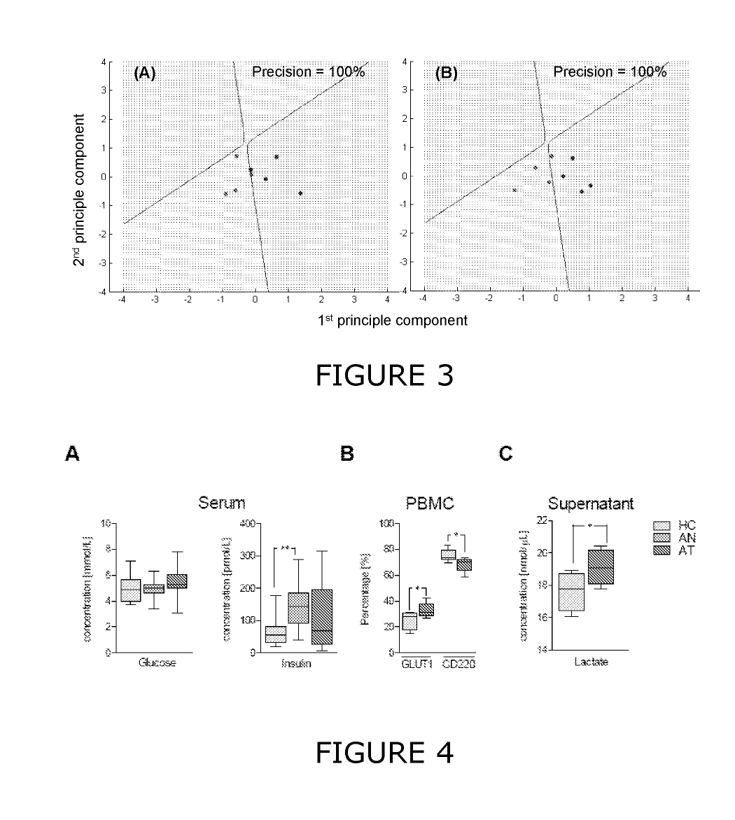

The same general result was obtained using sample sets of equal sizes (7 HC, 7 AN) to confirm that there was no bias introduced by different sample sizes (data not shown). This is also consistent with previous studies which have shown that glycolysis in immune cells is triggered after stimulation in vitro (Roos D, Loos J A. Biochimica et Biophysica Acta (BBA)-General Subjects 1970; 222(3): 565-82). Clusters involving enolase 1 (ENO1) and GAPDH gave high precision results for the stimulated samples although ENO1 was initially not found to be differentially expressed in patients. This demonstrates the power of the techniques for identifying molecules that exhibit patterned behaviour. The precision achieved for a similar cluster comprised of GAPDH and GPI is shown in FIG. 3. To confirm that these clusters were specific for the stimulated state, the top 5 clusters were chosen for stimulated PBMCs and were used to predict the diagnostic group of the unstimulated samples. This showed that the prediction results were consistently lower for unstimulated PBMCs (Table 4).

Functional Validation

Serum Analytes

Insulin signaling regulates glycolysis in most tissues (Wu et al, Experimental Gerontology 2005; 40(11): 894-9). As the majority of the differentially expressed proteins that we identified are associated with glycolysis, the circulating serum levels of insulin and glucose were measured in the same subjects from which the PBMCs were derived. Glucose levels were not significantly different between AN and HC subjects (p=0.3) and were in the normal range of glycemia (<7.8 mmol/L) (FIG. 4A). This is an important indicator since the patients were not fasted at the time of blood withdrawal. In contrast, insulin levels were increased 1.5-fold in AN patients compared to HC subjects (p=0.0013), confirming previous unpublished findings. Neither glucose nor insulin levels were significantly different in chronically ill AT patients.

PBMC Insulin Signalling Markers

Engagement of the T cell receptor (TCR) and co-ligation with CD28 leads to enhanced glucose transport and glycolysis (Frauwirth et al, Immunity 2002; 16(6): 769-77). In lymphocytes, the major glucose transporter and regulator of glucose uptake is GLUT1, an insulin-independent transporter that is up-regulated on the cell surface after T cell stimulation (Frauwirth et al, supra). The expression of GLUT1 and the insulin receptor (IR) was therefore analysed using stimulated PBMCs from 8 AN patients and 8 HC subjects (Table 1). The percentage of GLUT1-expressing PBMCs was increased 1.3-fold in AN patients when compared to HCs (p=0.022) and the percentage of PBMCs expressing the IR was decreased 1.1-fold in the same subjects (p=0.017) (FIG. 4B).

PBMC Glycolysis Markers

The alterations in GLUT1 expression described above, and the expression changes in glycolytic proteins are not sufficient to explain a functional change in glycolysis. Glucose uptake and the role of glucose within a cell are regulated by phosphorylation of glucose and this process is controlled by hexokinase. Therefore, the activity of this enzyme was measured in supernatants of lysed stimulated PBMCs obtained from 8 AN patients and 8 HC subjects (Table 1). No difference was observed in hexokinase activity between the two groups (p=0.9, data not shown). In addition, lactate levels were measured in cell supernatants of stimulated PBMCs since previous studies have shown that induction of glycolysis results in production and secretion of lactate from lymphocytes (Frauwirth K A, Thompson C B. J Immunol 2004; 172(8): 4661-5). FIG. 4C shows that lactate levels were significantly increased in AN patients compared to HC subjects (p=0.014).

### Discussion

The aim of this study was to identify altered proteomic signatures and molecular pathways to broaden our understanding of the pathophysiology underlying schizophrenia throughout different stages of the disease. PBMCs from first-onset AN and from chronically ill AT patients were profiled using a non-hypothesis driven LC-MS^{E} screening approach. It was important to include different patient subtypes since the aetiology and pathology are not known and the course of the disease may vary throughout life which is likely to be reflected by changes in molecular markers. Chronically ill AT patients are considered to be an established state of the illness whereas first-onset AN patients represent the first stages of disease without the confounding physiological effects of medication. The use of PBMCs for LC-MS^{E} profiling allowed downstream functional validation of protein hits and for identification of immunological and metabolic abnormalities.

Proteomic profiling of unstimulated PBMCs resulted in the identification of 6 proteins that were differentially expressed between the first onset patients and controls, whereas 13 differentially expressed proteins were identified after stimulation. Eight of these proteins were associated with glycolysis and were altered predominantly only in the case of the stimulated PBMCs. None of these proteins, with the exception of COTL1, showed differential expression in unstimulated PBMCs when comparing the chronically ill AT patients to controls, suggesting that at least some of these proteins may be normalized under long-term disease conditions or by treatment with antipsychotic medications. Therefore, further longitudinal studies should be carried out to determine whether these could be suitable as biomarkers for distinguishing patients from controls at the earliest stages of the disease or as responsive markers for monitoring antipsychotic treatment status. However, as sample numbers were low, it will be necessary to validate these findings using larger sample cohorts and samples from patients before and after drug treatment. Moreover, it would be of interest to investigate protein signatures in stimulated PBMCs obtained from various schizophrenia subtypes. The inventors have demonstrated that cellular/immunological conditions such as stimulation of PBMCs is recommended for these studies as this appeared to increase the separation between the disease and control states. These results highlight the importance of using cells for functional discovery of molecular pathways and demonstrate that it may not be sufficient to measure cellular protein expression levels in unstimulated states.

Here, protein clusters involving ENO1, GAPDH, GPI, PGM2 and TPIS, which are all key enzymes of the glycolysis pathway, resulted in the highest precision values for this separation between disease and control in stimulated cells.

Glycolysis provides the energy for immune cells to exert a full immune response (MacIver et al, J Leukoc Biol 2008; 84(4): 949-57) by acquiring metabolic substrates such as glucose from the circulation (Fox et al, Nat Rev Immunol 2005; 5(11): 844-52). However, immune cells are not capable of regulating the uptake of circulating metabolic substrates autonomously but instead this is controlled by hormones, cytokines or engagement of antigen and co-stimulatory receptors (Fox et al, supra). In this study, circulating glucose levels in first onset patients were relatively normal although insulin levels showed a significant elevation. This suggested that at least some of these patients were insulin resistant, consistent with recent unpublished findings. This means that the bioenergetic demands that maintain normal cellular functions in vivo, such as glucose uptake, activation of glycolysis and general regulation of insulin signalling, requires increased secretion of insulin from pancreatic β cells. This has important implications since numerous studies have suggested that too much insulin can have deleterious effects on brain function (Taguchi et al, Science 2007; 317(5836): 369-72). For example, hippocampal volumes appear to be reduced in diabetic patients and in insulin resistant individuals with high circulating insulin levels (Convit A. Neurobiol Aging 2005; 26 Suppl 1: 31-5). Also, hyperinsulinemia has been implicated in the pathogenesis of Alzheimer's disease and associated with phenomena such as aberrant phosphorylation of filamentous proteins, translocation of signalling molecules, increased central nervous system inflammation and β-amyloid plaque deposition (Craft S. Curr Alzheimer Res 2007; 4(2): 147-52).

The normal mechanism of stimulation by activation of the CD28 co-receptor triggers signaling cascades that overlap with those induced by binding of insulin to its receptor. In this case, stimulation resulted in increased expression of glycolytic proteins in first onset schizophrenia patients, along with increased numbers of GLUT1-expressing and decreased numbers of insulin receptor-expressing PBMCs. Although the increased GLUT1 expression suggests that glucose uptake might also be increased, it does not necessarily suggest an increase in glycolytic flux as this is controlled mainly by phosphorylation of hexokinase one of the main rate limiting enzymes in the glycolytic pathway (Frauwirth K A, Thompson C B. J Immunol, 2004; 172(8): 4661-5). Consistent with this, no change in hexokinase activity was found in the first onset patients. However, it is possible that the observed increase in expression of glycolytic proteins and increased production of lactate may compensate for perturbations of this pathway. A previous report showed decreased expression of glycolytic proteins at the transcriptomic level and increased lactate concentrations in the prefrontal cortex of schizophrenia subjects (Prabakaran et al, Mol Psychiatry 2004; 9(7): 684-97, 43).

Abnormalities in glucose metabolism and the link to metabolic syndrome in schizophrenia patients has been known for decades (J. M. Meyer SMS. Acta Psychiatrica Scandinavica 2009; 119(1): 4-14) with evidence deriving from genes associated with glycolysis and signs of abnormal glucose metabolism, including changes in glucose transporter expression (Stone et al, American Journal of Medical Genetics Part B: Neuropsychiatric Genetics 2004; 127B(1): 5-10). It has been believed for decades that the link between metabolic syndrome and schizophrenia derives solely from side-effects of antipsychotic medications (Dwyer et al, Ann Clin Psychiatry 2001; 13(2): 103-13). However, a study by Stone and co-workers supports the view that glycolytic abnormalities are inherent to the disease rather than deriving solely from environmental or pharmacological influences, as shown in this study (Stone et al, supra). The inventors have also shown that insulin levels are elevated prior to disease manifestation and it is demonstrated herein for the first time that patients' cells, most likely deriving from an insulin resistant environment, are compromised at the level of glycolysis when stimulated in vitro.

In conclusion, the inventors have found increased expression of proteins associated with the glycolysis pathway in first onset, antipsychotic naive schizophrenia patients. It has also been shown that small clusters of these proteins can be used to discriminate schizophrenia patients from control subjects with high precision. Most importantly, the alterations in glycolysis were seen to occur predominantly after PBMC stimulation as opposed to the situation in unstimulated cells. This highlights the importance of using functional cell investigations for mechanistic studies in schizophrenia. Using such approaches in future studies could help to identify and verify peripheral signatures and molecular pathways associated with schizophrenia and may generate much needed biomarkers for diagnostic and prognostic purposes and potentially facilitate the development of novel therapeutics.

What is claimed is:

1-6. (canceled)

7. A method of diagnosing or monitoring schizophrenia or predisposition thereto comprising detecting and/or quantifying, in a sample from a test subject, one or more analyte biomarkers selected from Cyclophilin A, Cytosolic non-specific dipeptidase, Coactosin-like protein, Glucose-6-phosphate isomerase, Uncharacterized protein KIAA0423, Myosin 14, Myosin 15, Nicotinamide phosphoribosyltransferase, Pyruvate kinase isozyme R/L and Phosphoglycerate mutase 4.

8. (canceled)

9. The method of claim 7, wherein samples are taken on two or more occasions from the test subject.

10. The method of claim 7, further comprising comparing the level of the one or more analyte biomarkers present in samples taken on two or more occasions.

11. The method of claim 7, further comprising comparing the amount of the one or more analyte biomarkers in said test sample with the amount present in one or more samples taken from said subject prior to commencement of therapy, one or more samples taken from said subject at an earlier stage of therapy, or both.

12. The method of claim 7, further comprising detecting a change in the amount of the one or more analyte biomarkers in samples taken on two or more occasions.

13. The method of claim 7, further comprising comparing the amount of the one or more analyte biomarkers present in said sample with one or more controls.

14. The method of claim 13, further comprising comparing the amount of the one or more analyte biomarkers in the sample with the amount of the biomarker present in a sample from a normal subject.

15. The method of claim 7, wherein samples are taken prior to and/or during and/or following therapy for schizophrenia.

16. The method of claim 7, wherein samples are taken at intervals over the remaining life, or a part thereof, of a subject.

17. The method of claim 7, wherein quantifying is performed by measuring the concentration of the one or more analyte biomarkers in the sample.

18. The method of claim 7, wherein detecting and/or quantifying is performed by one or more methods selected from SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

19. The method of claim 7, wherein detecting and/or quantifying is performed using an immunological method.

20. The method of claim 7, wherein the detecting and/or quantifying is performed using a biosensor or a microanalytical, microengineered, microseparation or immunochromatography system.

21. The method of claim 7, wherein the sample is cerebrospinal fluid, whole blood, blood serum, plasma, urine, saliva, or other bodily fluid, or breath, condensed breath, or an extract or purification therefrom, or dilution thereof.

22. (canceled)

23. A method of diagnosing or monitoring schizophrenia or predisposition thereto comprising detecting and/or quantifying, in a sample from a test subject, one or more analyte biomarkers selected from Cyclophilin A, Pyruvate kinase isozyme R/L, Phosphoglycerate mutase 4 and Glucose-6-phosphate isomerase.

24. The method of claim 7, further comprising detecting and/or quantifying, in the sample from the test subject one or more additional analyte biomarkers selected from L-lactate dehydrogenase B, Heat shock 70 kDa protein, Fructose bisphosphate aldolase, 60 kDa heat shock protein, Glyceraldehyde-3-phosphate dehydrogenase, Heterogeneous nuclear ribonucleoprotein, Phosphoglycerate kinase 1 and Triosephosphate isomerase.
